(19) United States
(12) Patent Application Publication
(10) Pub. No.: US 2008/0032965 A1
(43) Pub. Date: Feb. 7, 2008

(54) Method for enhancing cognitive function

(76) Inventors: Warren D. Hirst, Cranbury, NJ (US); Thomas A. Comery, Langhorne, PA (US); Suzan Aschmies, Trento, NJ (US); Sharon Joy Rosenzweig-Lipson, East Brunswick, NJ (US); Lee Erwin Schechter, Toms River, NJ (US)

Correspondence Address: WilmerHale/Wyeth, 60 STATE STREET, BOSTON, MA, 02109, US

(21) Appl. No.: 11/811,162

(22) Filed: Jun. 7, 2007

Related U.S. Application Data
(60) Provisional application No. 60/812,198, filed on Jun. 9, 2006.

(51) Int. Cl. A61K 31/496; A61K 31/435; A61K 31/451; A61P 25/28; A61K 31/55; A61K 31/495

(52) U.S. Cl. 514/215; 514/252.13; 514/252.17; 514/253.01; 514/255.03; 514/297; 514/319; 514/789

(57) ABSTRACT

Pharmaceutical compositions and compositions are provided for treating cognitive disorders using synergistically effective amounts of 5-HT_{1A} receptor antagonists and cognition enhancers.

This application claims the benefit of priority to U.S. Provisional Application No. 60/812,198, filed Jun. 9, 2006, the specification of which is incorporated by reference in its entirety

### FIELD OF THE INVENTION

The invention relates to methods for treating cognitive dysfunction. In particular the invention related to methods of treating cognitive dysfunction that include administration of a 5-HT_{1A} binding agent, in particular a 5-HT_{1A} receptor antagonist, in combination with a cognitive enhancer, and to pharmaceutical compositions containing synergistically effective amounts of a 5-HT_{1A} binding agent and a cognitive enhancer.

### BACKGROUND

Current therapies for cognitive dysfunction, e.g., cognitive deficits related to Alzheimer's disease, may exhibit undesired side effect profiles, such as liver damage, gastrointestinal problems (e.g., nausea, diarrhea, and vomiting), problems in digesting food, loss of appetite, abdominal pain, fatigue, and dizziness. Because of concerns regarding the severity or occurrence of side effects associated with these medications, combination therapies are typically not advised. Moreover, the positive cognitive effects observed with current therapies may not be sustained over a longer duration. Accordingly, there is a need to identify compounds and therapeutic regimens that are effective for ameliorating or preventing cognitive dysfunction, such as cognitive deficits associated with Alzheimer's disease. Similarly, there is a need to identify compounds and therapeutic regimens that have a lower risk of the occurrence of side effects.

### BRIEF DESCRIPTION OF THE INVENTION

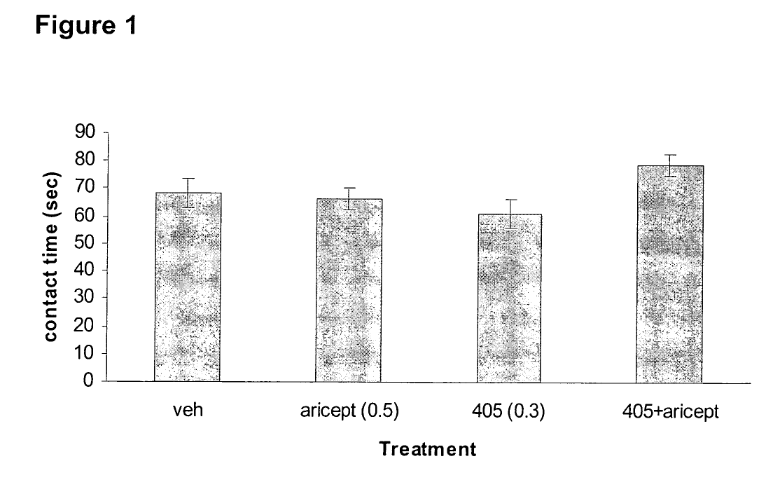

FIG. 1 is a tabular representation of the results of a Novel Object Recognition (NOR) test showing the differences in exploration between mice treated with either vehicle, 0.5 mg/kg of Aricept® alone (0.5), 0.3 mg/kg alone 405, and a combination of 405 and Aricept®.

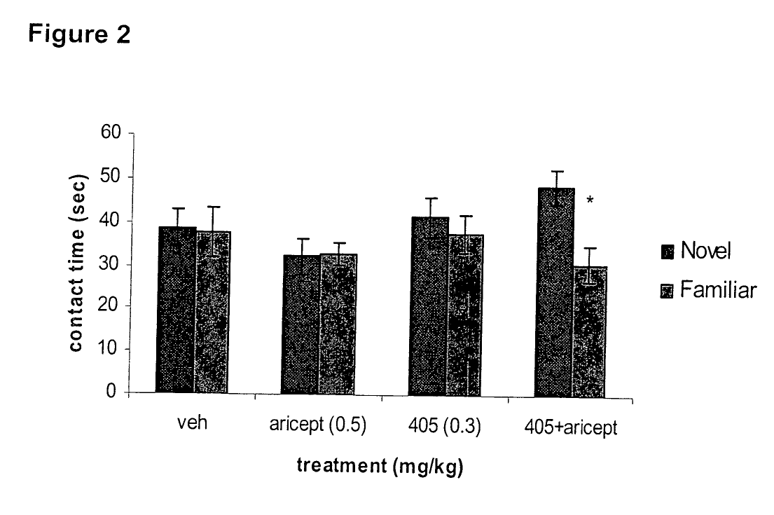

FIG. 2 is a tabular representation of the results of NOR tests showing the statistical differences in recognition and retention between mice treated with either vehicle, 0.5 mg/kg of Aricept® alone (0.5), 0.3 mg/kg alone 405, and a combination of 405 and Aricept®.

### DESCRIPTION OF THE INVENTION

It has been found that when compounds that are 5-HT_{1A} receptor antagonists are administered with cognitive enhancers, there is a synergistic effect on cognition enhancement. In some embodiments, the 5-HT_{1A} antagonist compound and the cognitive enhancer are administered in doses that are individually subtherapeutic. Thus, while 5-HT_{1A} antagonists have been investigated as potential therapeutics for a variety of conditions of the central nervous system, including cognitive dysfunction, it is surprising and unexpected that subtherapeutic doses of two agents operating along different physiological mechanisms will act synergistically to provide an enhancement in cognition.

Accordingly, the invention relates to a method for treating a cognitive disorder in a patient in need thereof. The method includes administering to the patient synergistically effective amounts of a compound that is a 5-HT_{1A} antagonist and a cognitive enhancer. In one embodiment, the amount of the cognitive enhancer administered to a patient in need thereof is less than an effective amount for enhancing cognition when administered separately from the 5-HT_{1A} antagonist. In one embodiment, the amount of the 5-HT_{1A} antagonist compound administered to a patient in need thereof is less than an effective amount for enhancing cognition when administered separately from the cognitive enhancer.

Cognitive disorders or cognitive dysfunction include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the present invention.

Compounds that are 5HT_{1A} antagonists are compounds that selectively bind to the 5-HT_{1A} receptor. More specifically, such compounds antagonize the activity of the 5-HT_{1A} receptor. Compounds that are 5-HT_{1A} antagonists can readily be identified by those skilled in the art using numerous art-recognized methods, including standard pharmacological test procedures such as those described herein.

The term “administer”, “administering”, or “administration”, as used herein refers to either directly administering a compound or pharmaceutically acceptable salt of the compound or a composition to an animal, or administering a prodrug derivative or analog of the compound or pharmaceutically acceptable salt of the compound or composition to the animal, which can form an equivalent amount of active compound within the animal's body.

The term “animal” as used herein includes, without limitation, a human, mouse, rat, guinea pig, dog, cat, horse, cow, pig, monkey, chimpanzee, baboon, or rhesus. In one embodiment, the animal is a mammal. In another embodiment, the animal is a human.

In one embodiment, compounds that are 5-HT_{1A} antagonists include compounds described in U.S. Pat. Nos. 6,127,357 and 6,465,482, U.S. application Ser. No. 11/396,307, entitled “Serotonergic Agents For Treating Sexual Dysfunction”, filed Mar. 30, 2006, U.S. application Ser. No. 11/450,942, entitled “Piperazine-Piperidine Antagonists And Agonists Of The 5-HT_{1A} Receptor”, filed on Jun. 9, 2006, and International Patent Publication Nos. WO 97/03982, and WO 95/33743, all of which are incorporated by reference. These compounds can be prepared according to the methods described in these patents and patent publications. In one embodiment, 5-HT_{1A} antagonists include compounds described in Caliendo, et al. “Derivatives as 5HT_{1A} Receptor Ligands—Past and Present”, Current Medicinal Chemistry, 12:1721-1753 (2005), which is incorporated by reference. Nonlimiting examples of 5-HT_{1A} antagonists described by Caliendo, et al. include aminotetralins (e.g., S-UH301 and 5-Me-OH-DPAT), ergolines, arylpiperazines (e.g., SDZ 216,525, DU 125530, DU 125430, compounds 100-106 in Table 1, compounds 124, 125, 127 and 128 in Table 2, compound 114 in Table 3, and compounds 131 and 132 in Table 4), indolylalkylamines, apophines (e.g., Compound A), and aryloxyalkylamines.

TABLE 1

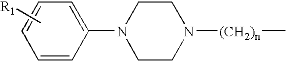

| | | | | Ki (nM) | |
|---|---|---|---|---|---|
| Compd. | R₁ | n | R | 5-HT_{1A} | 5-HT_{2A} |
| 99 | 3-Cl | 3 | A | 253 | 16 |
| 100 | 3-Cl | 4 | A | 50 | 68 |
| 101 | 2-OCH₃ | 4 | A | 36 | 566 |
| 102 | 2-OCH₃ | 3 | B | 30 | 300 |
| 103 | 2-OCH₃ | 4 | B | 43 | 375 |
| 104 | 3-Cl | 4 | C | 50 | 1830 |
| 105 | 2-OCH₃ | 3 | C | 54 | 2120 |
| 106 | 2-OCH₃ | 4 | C | 51 | 1450 |

| | |
|---|---|
| R = | A |
| 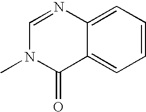 | |
| 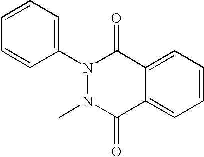 | B |
| 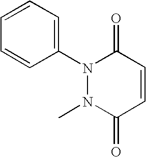 | C |

TABLE 2

| | | Ki (nM) | | |
|---|---|---|---|---|
| Compd. | Structures | 5-HT_{1A} | D₂ | a1 |
| Compound Y | 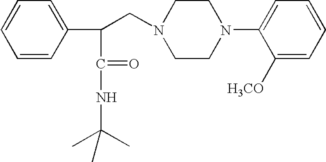 | (±) 20 (S) 22 (R) 104 | (±) 14 (S) 20 (R) 4 | (±) 718 (S) 1440 (R) 1240 |
| 118 | 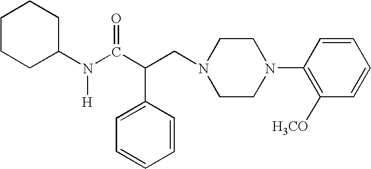 | 10 | — | 1870 |
| Compound X | 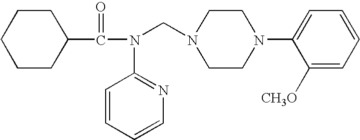 | 0.24 | 79 | 45 |
| 119 R = H | 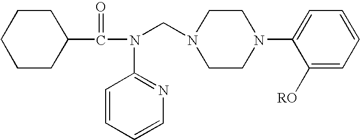 | 2.10* | | |
| 120 R = (CH₂)₂F | | 1.55 | | |
| 121 R = (CH₂)₃F | | 6.05 | — | — |
| 122 R = SO₂CH₃ | | 7.25 | | |
| 123 R = SO₂CF₃ | | 36 | | |
| 124 (p-MPPI) R = I | 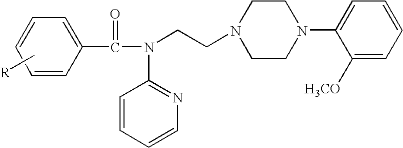 | 2.6 | 19 | 35 |
| 125 (m-MPPI) R = I | | 1.7 | | |
| 126 (o-MPPI) R = I | | 10.4 | | |
| 127 (p-MPPF) R = F | | 3.3 | | |
| 128 (p-MPPN) R = NO₂ | | 1.6 | | |

Data is expressed as *IC₅₀ values.

TABLE 3

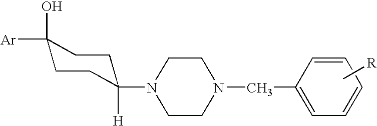

| | | | IC₅₀ (nM) |
|---|---|---|---|
| Compd | Ar | R | 5-HT_{1A} |
| 112 | 3,4-(OCH₂O-)-Ph | H | 20 |
| 113 | 4-OCH₃-Ph | H | 50 |
| 114 | 3,4-(OCH₂O-)-Ph | 3-OCH₃ | 2.2 |

TABLE 4

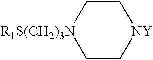

| | | | | | Ki (nM) | |
|---|---|---|---|---|---|---|
| Compd | R₁ | R₅ | R₆ | Y | 5-HT_{1A} | *1^{A} |
| 129 | 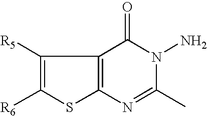 | —(CH₂)₃— | | C₆H₄OCH₃(o) | 9.26 | 7.39 |
| 130 | | H | C₃H₇ | C₆H₄OCH₃(o) | 1.40 | 19.65 |
| 131 | | H | C₂H₅ | C₆H₄NO₂(o) | 9.69 | 194.8 |
| 132 | | H | H | C₆H₄NO₂(o) | 3.0 | 39.87 |
| 133 | | H | C₂H₅ | 2-pyrimidinyl | 9.40 | 1946 |
| 134 | | H | H | 2-pyrimidinyl | 5.06 | 366.31 |
| 135 | 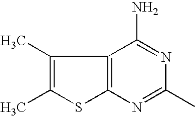 | | | C₆H₄OCH₃(o) | 2.45 | 10.8 |
| 136 | | | | C₆H₄OCH₃(o) | 6.84 | 31.50 |
| 137 | 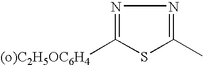 | | | C₆H₄NO₂(o) | 135 | 437 |

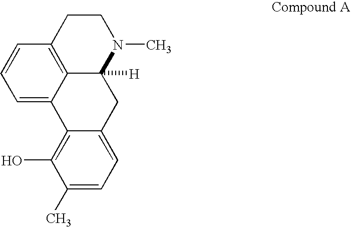

Non-limiting examples of compounds useful in the invention include, without limitation:

(R)-4-cyano-N-{2-[4-(2,3-dihydro-benzo[1,4]dioxin-5-yl)-piperazin-1-yl]propyl}-N-pyridin-2-yl-benzamide (lecozotan) and pharmaceutically acceptable acid addition salts thereof,

N-[2-[4-(2-methoxyphenyl)-1-piperazinyl]ethyl]-N-(2-pyridinyl)cyclohexanecarboxamide and pharmaceutically acceptable acid addition salts thereof,

(R)-N-(2-methyl-(4-indolyl-1-piperazinyl)ethyl)-N-(2-pyridinyl)cyclohexane carboxamide (Compound 405) and pharmaceutically acceptable acid addition salts thereof,

5-fluoro-8-(4-(4-(6-methoxyquinolin-8-yl)piperazin-1-yl)piperidin-1-yl)quinoline or a pharmaceutically acceptable acid addition salt thereof,

5-fluoro-4-methoxy-8-(4-(4-(6-methoxyquinolin-8-yl)piperazin-1-yl)piperidin-1-yl)-2-(trifluoromethyl)quinoline and pharmaceutically acceptable acid addition salts thereof,

6-methoxy-8-[4-(1-quinolin-8-yl-piperidin-4-yl)-piperazin-1-yl]-quinoline and pharmaceutically acceptable acid addition salts thereof,

6-fluoro-8-[4-(1-quinolin-8-yl-piperidin-4-yl)-piperazin-1-yl]-quinoline and pharmaceutically acceptable acid addition salts thereof,

8-{4-[4-(1H-indole-4-yl)-piperazin-1-yl]-piperidin-1-yl}-quinoline and pharmaceutically acceptable acid addition salts thereof,

5-fluoro-8-{4-[4-(5-fluoro-benzofuran-3-yl)-piperazin-1-yl]- piperidin-1-yl}-quinoline and pharmaceutically acceptable acid addition salts thereof,

7-fluoro-8-(4-(4-(6-methoxyquinolin-8-yl)piperazin-1-yl)piperidin-1-yl)quinoline and pharmaceutically acceptable acid addition salts thereof,

6-methoxy-8-(4-(1-(quinolin-8-ylmethyl)piperidin-4-yl)piperazin-1-yl)quinoline and pharmaceutically acceptable acid addition salts thereof,

6-methoxy-8-(4-(1-(quinolin-8-ylmethyl)piperidin-4-yl)piperazin-1-yl)quinoline and pharmaceutically acceptable acid addition salts thereof,

8-[4-(1-quinolin-8-yl-piperidin-4-yl)-piperazin-1-yl]-5-trifluoromethyl-quinoline and pharmaceutically acceptable acid addition salts thereof,

5-methoxy-8-[4-(1-quinolin-8-yl-piperidin-4-yl)-piperazin-1-yl]-quinoline and pharmaceutically acceptable acid addition salts thereof,

5-fluoro-8-[4-(4-quinolin-8-yl-piperazin-1-yl)-piperidin-1-yl]-quinoline and pharmaceutically acceptable acid addition salts thereof, and

8-[4-(4-benzofuran-3-yl-piperazin-1-yl)-piperidin-1-yl]-6-chloro-quinoline and pharmaceutically acceptable acid addition salts thereof.

In certain embodiments, the 5-HT_{1A} antagonist compound is any one of the following:

(R)-4-cyano-N-{2-[4-(2,3-dihydro-benzo[1,4]dioxin-5-yl)-piperazin-1-yl]propyl}-N-pyridin-2-yl-benzamide (lecozotan) and pharmaceutically acceptable acid addition salts thereof,

N-[2-[4-(2-methoxyphenyl)-1-piperazinyl] ethyl]-N-(2-pyridinyl)cyclohexanecarboxamide and pharmaceutically acceptable acid addition salts thereof,

(R)-N-(2-methyl-(4-indolyl-1-piperazinyl)ethyl)-N-(2-pyridinyl) cyclohexanecarboxamide and pharmaceutically acceptable acid addition salts thereof,

6-methoxy-8-[4-(1-quinolin-8-yl-piperidin-4-yl)-piperazin-1-yl]-quinoline and pharmaceutically acceptable acid addition salts thereof,

6-fluoro-8-[4-(1-quinolin-8-yl-piperidin-4-yl)-piperazin-1-yl]-quinoline and pharmaceutically acceptable acid addition salts thereof.

In particular embodiments, the 5-HT_{1A} antagonist compound is

(R)-4-cyano-N-{2-[4-(2,3-dihydro-benzo[1,4]dioxin-5-yl)-piperazin-1-yl]propyl}-N-pyridin-2-yl-benzamide (lecozotan) and pharmaceutically acceptable acid addition salts thereof,

N-[2-[4-(2-methoxyphenyl)-1-piperazinyl]ethyl]-N-(2-pyridinyl)cyclohexane carboxamide and pharmaceutically acceptable acid addition salts thereof,

(R)-N-(2-methyl-(4-indolyl-1-piperazinyl)ethyl)-N-(2-pyridinyl)cyclohexane carboxamide and pharmaceutically acceptable acid addition salts thereof, or

6-methoxy-8-[4-(1-quinolin-8-yl-piperidin-4-yl)-piperazin-1-yl]-quinoline and pharmaceutically acceptable acid addition salts thereof.

In more particular embodiments, the 5-HT_{1A} antagonist compound is either

(R)-4-cyano-N-{2-[4-(2,3-dihydro-benzo[1,4]dioxin-5-yl)-piperazin-1-yl]propyl}-N-pyridin-2-yl-benzamide (lecozotan) and pharmaceutically acceptable acid addition salts thereof, or

(R)-N-(2-methyl-(4-indolyl-1-piperazinyl)ethyl)-N-(2-pyridinyl)cyclohexane carboxamide and pharmaceutically acceptable acid addition salts thereof.

In the most particular embodiment, the 5-HT_{1A} antagonist compound is

(R)-4-cyano-N-{2-[4-(2,3-dihydro-benzo[1,4]dioxin-5-yl)-piperazin-1-yl]propyl}-N-pyridin-2-yl-benzamide (lecozotan) and pharmaceutically acceptable acid addition salts thereof.

The pharmaceutically acceptable salts are generally the acid addition salts which can be formed from a compound of a general formula described herein and a pharmaceutically acceptable acid such as, for example, benzoic, phosphoric, sulfuric, hydrochloric, hydrobromic, citric, maleic, malic, mandelic, mucic, nitric, fumaric, succinic, tartaric, acetic, lactic, pamoic, pantothenic, benzenesulfonic, adipic or methanesulfonic acid. In some embodiments of the invention the acid addition salt is hydrochloric acid or succinic acid. Other pharmaceutically acceptable salts known to those in the art can be used.

Cognitive enhancers that are co-administered with the 5-HT_{1A} antagonist compounds of this invention include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Publication Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5-HT receptor modulators other than 5-HT_{1A} antagonists (examples of 5-HT receptor modulators other than 5-HT_{1A} antagonists include without limitation, 5-HT₆ antagonists (nonlimiting examples of such compounds are described in U.S. Pat. Nos. 6,727,236, 6,825,212, 6,995,176, and 7,041,695))); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) inhibitors (e.g., PDE IV inhibitors, roflumilast, arofylline, cilomilast, rolipram, RO-20-1724, theophylline, denbufylline, ARIFLO, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem camosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

“Provided,” as used herein with respect to providing a compound or substance covered by this invention, means either directly administering such a compound or substance, or administering a prodrug, derivative, or analog which will form an equivalent amount of the compound or substance within the body. Prodrugs can be prepared such as described in Design of Prodrugs, Bundgaard, H. ed., (Elsevier, New York 1985); Prodrugs as Novel Drug Delivery Systems, Higuchi, T and Stella, V. eds, (American Chemical Society, Washington, D.C. 1975); Design of Biopharmaceutical Properties through Prodrugs and Analogs, Roche, E. ed., (American Pharmaceutical Association Academy of Pharmaceutical Sciences, Washington, D.C., 1977); and Metabolic Considerations in Prodrug Design, Balant, L.P. and Doelker, E. in Burger's Medicinal Chemistry and Drug Discovery, Fifth Edition, Wolff, M., ed, Volume 1, pages 949-982, (John Wiley & Sons, Inc. 1995).

Compounds, including 5-HT_{1A} antagonist compounds and cognitive enhancers, as described herein are useful for the preparation of a medicament for use in treating a cognitive disorder or for use in enhancing cognition.

As used in this invention, the combination regimen can be given simultaneously or can be given in a staggered regimen, with the 5-HT_{1A} antagonist compound or pharmaceutical salt of the 5-HT_{1A} antagonist compound being given at a different time than the cognitive enhancer. This time differential may range from several minutes, hours, days, weeks, or longer between administration of the two agents. Therefore, the term combination does not necessarily mean administered at the same time or as a unitary dose, but that each of the components are administered during a desired treatment period. The agents may also be administered by different routes. For example, in the combination of the 5-HT_{1A} antagonist compound, or pharmaceutical salt thereof, plus the cognitive enhancer, it is anticipated that the 5-HT_{1A} antagonist compound, or pharmaceutical salt thereof, will be administered orally or parenterally, while the cognitive enhancer may be administered parenterally, orally, or by other acceptable means. These combinations can be administered hourly, daily, weekly, or even once monthly.

It is understood that the dosage of each component in the combination regimen may vary depending on the particular compound utilized, the mode of administration, the condition, and severity thereof, of the condition being treated, as well as the various physical factors related to the individual being treated. Moreover, it is understood that the effective dosage of the combination may vary depending on the particular compound utilized, the mode of administration, the condition, and severity thereof, of the condition being treated, as well as the various physical factors related to the individual being treated.

The amount of the effective dosage of the combination of a 5-HT_{1A} antagonist compound, or a pharmaceutically acceptable salt of a 5-HT_{1A} antagonist compound, and a cognitive enhancer is an amount that is effective for treating or preventing a cognitive disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner.

The amount of the 5-HT_{1A} compound or a pharmaceutically acceptable salt of the compound administered as part of the combination regimen will typically range from about 0.001 mg/kg to about 600 mg/kg of body weight per day, in one embodiment, less than about 600 mg/kg body weight per day, in another embodiment, less than about 400 mg/kg body weight per day, in another embodiment, less than about 200 mg/kg of body weight per day, in another embodiment, less than about 100 mg/kg of body weight per day, in another embodiment, less than about 10 mg/kg body weight per day, in another embodiment, less than about 1 mg/kg of body weight per day, in another embodiment, less than about 0.5 mg/kg of body weight per day, in another embodiment, less than about 0.1 mg/kg of body weight per day, and in another embodiment, and less than about 0.001 mg/kg of body weight per day. In one embodiment, the amount of the 5-HT_{1A} compound or a pharmaceutically acceptable salt of the compound administered as part of the combination regimen is less than its effective amount would be where the other cognitive enhancer is not administered. Thus, the amount of the 5-HT_{1A} compound or a pharmaceutically acceptable salt of the compound is a subtherapeutic amount or a subthreshold amount. In this case, without being bound by theory, it is believed that the 5-HT_{1A} compound or a pharmaceutically acceptable salt of the compound and the cognitive enhancer act synergistically. In some cases, the patent in need of treatment is being treated with one or more other therapeutic agents.

The amount of the cognitive enhancer in the combination will typically range from about 0.001 mg/kg to about 600 mg/kg of body weight per day, in one embodiment, less than about 600 mg/kg body weight per day, in another embodiment, less than about 400 mg/kg body weight per day, in another embodiment, less than about 200 mg/kg of body weight per day, in another embodiment, less than about 100 mg/kg of body weight per day, in another embodiment, less than about 10 mg/kg body weight per day, in another embodiment, less than about 1 mg/kg of body weight per day, in another embodiment, less than about 0.5 mg/kg of body weight per day, and in another embodiment, less than about 0.1 mg/kg of body weight per day, in another embodiment, less than about 0.001 mg/kg of body weight per day. In one embodiment, the amount of cognitive enhancer administered as part of the combination regimen is less than its effective amount would be where the 5-HT_{1A} compound or a pharmaceutically acceptable salt of the compound is not administered. Thus, the amount of the cognitive enhancer is a subtherapeutic amount or a subthreshold amount. In this case, without being bound by theory, it is believed that the 5-HT_{1A} compound or a pharmaceutically acceptable salt of the compound and the cognitive enhancer act synergistically. In some cases, the patent in need of treatment is being treated with one or more other therapeutic agents.

As discussed herein, the pharmaceutical dosage form can be a dosage form comprising either a 5-HT1A antagonist compound, or a pharmaceutical salt of a 5-HT1A antagonist compound, or a cognitive enhancer, or both a 5-HT1A antagonist compound, or a pharmaceutical salt of a 5-HT1A antagonist compound, and a cognitive enhancer. Similarly, as discussed herein, the pharmaceutical composition can be either agent separately or the two agents together in one composition.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 0.001 mg/kg to about 250 mg/kg of the 5-HT_{1A} compound or a pharmaceutically acceptable salt of the compound, and may be given in a single dose or in two or more divided doses. Similarly, such unit dosage form may contain from about 0.001 mg/kg to about 250 mg/kg of the cognitive enhancer, and may be given in a single dose or in two or more divided doses. Variations in the dosage will necessarily occur depending upon the species, weight and condition of the patient being treated and the patient's individual response to the medicament.

In one embodiment, the unit dosage form is about 0.001 to about 1000 mg of the 5-HT_{1A} antagonist compound or a pharmaceutically acceptable salt of the 5-HT_{1A} antagonist compound. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg.

In one embodiment, the unit dosage form is about 0.001 to about 1000 mg of the cognitive enhancer. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg.

When administered to an animal, the compounds or pharmaceutically acceptable salts of the compounds can be administered neat or as a component of a composition that comprises a physiologically acceptable carrier or vehicle. A pharmaceutical composition of the invention can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, excipient, or diluent.

The present pharmaceutical compositions, comprising compounds or pharmaceutically acceptable salts of the compounds of the invention, can be administered orally. The compound of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound or a pharmaceutically acceptable salt of the compound into the bloodstream. The mode of administration is left to the discretion of the practitioner.

In one embodiment, the compound of the invention is administered orally.

In another embodiment, the compound of the invention is administered intravenously.

In another embodiment, it may be desirable to administer the compound of the invention locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the compound of the invention into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound or a pharmaceutically acceptable salt of the compound can be formulated as a suppository, with traditional binders and excipients such as triglycerides.

In another embodiment, the compound of the invention can be delivered in a vesicle, in particular a liposome (see Langer, Science 249:1527-1533 (1990) and Treat et al., Lipsomes in the Therapy of Infectious Disease and Cancer 317-327 and 353-365 (1989)).

In yet another embodiment, the compound of the invention can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-)1990); Sefton, CRC Crit. Ref. Biomed. Eng. 14:201 (1987); Buchwald et al., Surgery 88:507 (1980); and Saudek et al., N. Engl. J Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to an animal. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The compound or pharmaceutically acceptable salt of the compound of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences 1447-1676 (Alfonso R. Gennaro, ed., 19th ed. 1995).

In one embodiment, the compound or a pharmaceutically acceptable salt of the compound is formulated, in accordance with routine procedures, as a composition adapted for oral administration to humans. Compositions for oral delivery can be in the form of tablets, lozenges, buccal forms, troches, aqueous or oily suspensions or solutions, granules, powders, emulsions, capsules, capsules containing liquids, syrups, aerosols, sprays, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of the compounds or pharmaceutically acceptable salts of the compounds with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.) Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the compound or a pharmaceutically acceptable salt of the compound can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the compound or a pharmaceutically acceptable salt of the compound is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

In another embodiment, the compound or pharmaceutically acceptable salt of the compound can be administered transdermally through the use of a transdermal patch. Transdermal administrations include administrations across the surface of the body and the inner linings of the bodily passages including epithelial and mucosal tissues. Such administrations can be carried out using the present compounds or pharmaceutically acceptable salts of the compounds, in lotions, creams, foams, patches, suspensions, solutions, and suppositories (e.g., rectal or vaginal).

Transdermal administration can be accomplished through the use of a transdermal patch containing the compound or pharmaceutically acceptable salt of the compound and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the active ingredient.

The compounds or pharmaceutically acceptable salts of the compounds of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The compound or a pharmaceutically acceptable salt of the compound can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of the compound or a pharmaceutically acceptable salt of the compound to treat or prevent a cognitive disorder in a minimal amount of time. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, the present invention is directed to prodrugs of the compounds or pharmaceutically acceptable salts of compounds of the present invention. Various forms of prodrugs are known in the art, for example, as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs,” Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

In one aspect the invention includes a pharmaceutical product containing a 5-HT_{1A} antagonist and a cognitive enhancer as a combined preparation for simultaneous, separate or sequential use in therapy for treating a cognitive disorder. In another aspect the invention includes use of a 5-HT_{1A} antagonist and a cognitive enhancer in the manufacture of a medicament for treating a cognitive disorder. In another aspect the invention includes use of a cognitive enhancer in the manufacture of a medicament, for use with a 5-HT_{1A} antagonist, for treating a cognitive disorder. In another aspect the invention includes use of a 5-HT_{1A} antagonist in the manufacture of a medicament, for use with a cognitive enhancer, for treating a cognitive disorder.

### EXAMPLES

The present invention is illustrated by reference to the following examples and additional information. The examples of experiments are provided for illustrative purposes only. They are not to be construed as limiting the scope or content of the invention in any way. Those skilled in the art of organic synthesis may be aware of still other synthetic routes to the invention compound. The reagents and intermediates used herein are either commercially available or prepared according to standard literature procedures.

Methods of testing the effect of an invention compound on cognitive dysfunction are described infra. Such methods are useful for identifying 5-HT_{1A} antagonists (i.e., 5-HT_{1A} receptor antagonists) and cognitive enhancers that are effective for treating a cognitive disorder. Other methods of testing the effect of a compound on cognitive dysfunction are known in the art and include, for example the following protocols: contextual or cued fear conditioning (Comery, T. A. et al., Journal of Neuroscience 25(39): 8898-8902 (September 28, 2005)), passive avoidance (Foley, A. G. et al., Neuropsychopharmacology 29: 93-100 (2004)), radial arm maze (Boast, C. et al., Neurobiology of Learning and Memory 71: 259-271 (1999)), morris water maze (Day, M. and Langston, R. F., Neuroscience 137: 19-28 (2006)), and 5-choice serial reaction task (Robbins, T. W., Psychopharmacology 163: 362-380 (2002)).

### Example 1

### Cognition Enhancement—Novel Object Recognition

Acetylcholinesterase inhibitors are currently used to treat mild cognitive deficits resulting from Alzheimer's disease. However, the use of acetylcholinesterase inhibitors is hindered by the side effects often seen with this class of therapeutic agent, and have limited efficacy. 5-HT_{1A} antagonists (5-fluoro-4-methoxy-8-(4-(4-(6-methoxyquinolin-8-yl)piperazin-1-yl)piperidin-1-yl)-2-(trifluoromethyl)quinoline AND 5-fluoro-8-(4-(4-(6-methoxyquinolin-8-yl)piperazin-1-yl)piperidin-1-yl)quinoline) were shown to have a synergistic effect on the cognitive enhancing properties of a representative acetylcholinesterase inhibitor, Aricept®, lowering the dose of both agents required to obtain efficacy in the Novel Object Recognition Model.

Novel Object Recognition Model: Male Long-Evans hooded rats (˜200 g at the time of testing) were individually housed with ad libitum access to food and water. Novel object recognition (NOR) training and testing was performed in a circular field (diameter ˜70 cm, 30 cm high) constructed out of plastic and containing soiled bedding (without feces). The field was surrounded by black curtains to mask extra-field cues and was located in a dimly lit room (˜10 lux at the level of the area) in the presence of white noise (˜65 dB). Animal performance was tracked by video and monitored by an experimenter located outside of the testing room. Objects, constructed with Duplo® blocks (Lego), could be affixed to the floor in one of four locations spaced evenly around the field approximately 10 cm from the field's edge. To avoid possible olfactory cues, multiple copies of the objects were used throughout the study and were cleaned with a 30% ethanol solution between animals.

The visual recognition task was divided into 3 sessions—habituation, a sample trial and a choice trial. During habituation the animals were placed into the field containing 2 identical yellow cubes (˜10 cm×10 cm×10 cm) and were allowed to explore the field for ten minutes. Following habituation, rats were returned to their home cage. One day after habituation, animals were dosed with drug (either a 5-HT_{1A} antagonist compound, a cognitive enhancer or both a 5-HT_{1A} antagonist compound and a cognitive enhancer) and following the pretreatment interval the sample trial was initiated. During the sample trial, rats were allowed to explore the field, now containing two identical stimuli (complex, multicolored, Duplo objects; ˜10cm×˜10cm×˜10 cm) located at opposing compass points, for 5 minutes. The amount of time investigating the objects was recorded for the entire trial. Investigation was defined as orientation toward the object with the nose of the rat within <2 cm of the object. Following the sample trial rats were returned to their home cages for the 48 hour inter-trial interval and then tested in the choice trial for recognition memory. The choice trial consisted of a 5 minute exploration of the field containing both a familiar, previously explored, object and a novel object with an investigator again recording contact time. The location of the objects, counterbalanced across treatment groups, remained constant for each animal during the habituation, sample and choice trials.

The effect of treatment on object exploration during trial one was examined using a one-way ANOVA on total contact time followed by Fisher's LSD group mean pair-wise comparisons. The amount of time exploring the novel and familiar objects across treatment groups was analyzed using a repeated measures ANOVA followed by Fisher's LSD post-hoc comparisons. Significantly more time spent exploring the novel object than the familiar one represents intact recognition memory for that treatment group. Control and untreated animals show no significant differences between familiar and novel object exploration following the 48 hour delay indicating no memory for the sample trial (significant differences are evident with shorter delays).

Sub-threshold doses (doses that did not provide a positive effect on recognition memory) of Aricept® and 5-HT_{1A} antagonist compounds were administered to test animals and their effects on recognition memory were recorded as described above.

Treatments: Animals were treated with a 5-HT_{1A} antagonist compound 60 minutes prior to the sample trials. Each candidate compound was dissolved in an appropriate vehicle and administered orally. The same animals were then treated with Aricept® 30 minutes prior to the sample trials. Aricept® was dissolved in an appropriate vehicle and administered intraperitoneally.

Animals were separately administered sub-threshold doses of Aricept® (0.5 mg/kg i.p.) or a 5-HT_{1A} antagonist compound (0.1 mg/kg p.o.). When administered separately, the contact times for the animals in the familiar and novel environments were not statistically different (P<0.05). Co-administration of Aricept® and each of the 5-HT_{1A} antagonist compounds at the same doses used in the separate administration test for each agent resulted in a statistically significant increase in the contact time for the novel environment compared to the familiar environment (P<0.05). The increase in contact time averaged more than 10 seconds. These data suggest co-administration of a 5-HT_{1A} antagonist compound and a cognitive enhancer resulted in a positive effect of recognition memory, as demonstrated by the animals spending significantly more time exploring the novel object than the familiar one. These data demonstrate that this test is effective in identifying a synergistic effect between 5-HT_{1A} antagonist compounds and cognitive enhancers to enhance cognition. These data also demonstrate that the 5-HT_{1A} antagonist compounds act synergistically with cognitive enhancers to enhance cognition.

The Examples provided supra illustrate methods that can be used to test agents described herein for their ability to ameliorate cognitive dysfunction. Other models known in the art for testing cognitive dysfunction can be used.

### Example 2

### Cognition Enhancement using the 5-HT_{1A} Antagonist (R)-N-(2-methyl-(4-indolyl-1-piperazinyl)ethyl)-N-(2-pyridinyl) cyclohexane carboxamide

Cognitive enhancement was measured using the Novel Object Recognition procedure described in Example 1. However, there were some modifications. For instance, the 5-HT_{1A} antagonist, (R)-N-(2-methyl-(4-indolyl-1-piperazinyl)ethyl)-N-(2-pyridinyl)cyclohexane carboxamide (Compound 405), was used in dosages of 0.3 mg/kg animal weight. Furthermore, the pretreatment period for Compound 405 was 120 minutes, rather than 60 minutes.

The results showed that exploration time of the mice was relatively similar between mice treated with vehicle, those treated with Aricept® alone, mice treated with Compound 405 alone, and mice treated with combination therapy consisting of Aricept® and Compound 405 (FIG. 1). Mice treated with combination therapy comprising Aricept® and Compound 405 showed significantly improved retention and recognition (FIG. 2). When exposed to familiar surroundings, the combination therapy group showed statistically significant reduction in contact time as compared to the groups treated with vehicle or with individual doses of either Aricept® or Compound 405 (FIG. 2).

### Example 3

### In Vitro Activity

The following protocols are effective for demonstrating the ability of candidate compounds to bind to the 5-HT_{1A} receptor. The protocols are also effective for demonstrating antagonistic effects of candidate compounds.

### Cell Line

The PCR cloning of the human 5-HT_{1A} receptor subtype from a human genomic library has been described previously (Chanda et al., Mol. Pharmacol., 43:516 (1993)). A stable Chinese hamster ovary cell line expressing the human 5-HT_{1A} receptor subtype (h5-HT_{1A}.CHO cells) is employed throughout this study. Cells are maintained in DMEM supplemented with 10% fetal calf serum, non-essential amino acids and penicillin/ streptomycin.

### Radioligand Binding

Radioligand binding assays are performed as described in Dunlop, J. et al., J. Pharmacol. and Toxicol. Methods 40: 47-55 (1998), which is incorporated by reference. Cells are grown to 95-100% confluency as a monolayer before membranes were harvested for binding studies. Cells are gently scraped from the culture plates, transferred to centrifuge tubes, and washed twice by centrifugation (2000 rpm for 10 min., 4° C.) in buffer (50 mM Tris; pH 7.5). The resulting pellets are aliquoted and placed at −80° C. On the day of assay, the cells are thawed on ice, and resuspended in buffer. Studies are conducted using [³H]8-OH-DPAT as the radioligand. The binding assay is performed in 96 well microtiter plates in a final total volume of 250 μL of buffer. Competition experiments are performed by using seven different concentrations of unlabelled drug and a final ligand concentration of 1.5 nM. Non-specific binding is determined in the presence of 10 μM 5HT. Saturation analysis is conducted by using [³H]8-OH-DPAT at concentrations ranging from 0.3-30 nM. Following a 30 minute incubation at room temperature, the reaction is terminated by the addition of ice cold buffer and rapid filtration using a M-96 Brandel Cell Harvester (Gaithersburg, Md.) through a GF/B filter presoaked for 30 minutes in 0.5% polyethyleneimine.

### cAMP Measurements

Measurements are performed as described in Dunlop, J. et al., supra. Assays are performed by incubating the cells with DMEM containing 25 mM HEPES, 5 mM theophylline and 10 μM pargyline for a period of 20 minutes at 37° C. Functional activity is assessed by treating the cells with forskolin (1 uM final concentration) followed immediately by test compound (6 different concentrations) for an additional 10 minutes at 37° C. In separate experiments, 6 concentrations of antagonist are preincubated for 20 minutes prior to the addition of 10 nM 8-OH-DPAT and forskolin. The reaction is terminated by removal of the media and addition of 0.5 ml ice cold assay buffer. Plates are stored at −20° C. prior to assessment of cAMP formation by a cAMP SPA assay (Amersham).

The protocol is effective for identifying compounds that have 5-HT_{1A} agonist activity and 5-HT_{1A} antagonist activity. 5-HT_{1A} agonist activity is demonstrated by inhibiting the forskolin-induced increase in cAMP levels and the results reported as EC₅₀ values. Compounds having 5-HT_{1A} antagonist activity show no effect on forskolin-induced increases in cAMP levels on their own, but block the 8-OH-DPAT-induced inhibition of forskolin-stimulated increases in cAMP levels. Results are required as IC₅₀ values.

### Example 4

### In Vivo Functional Activity

The ability of the compounds to function in vivo as 5-HT_{1A} antagonists can be assessed in rats using a Fixed Responding Model (D. Blackman, in “Operant Conditioning: An Experimental Analysis of Behavior,” J. Butcher, ed., Methuen and Co., Ltd., London). In this model rats are trained to respond (lever pressing) under a fixed-ratio 30 schedule of food presentation in order to receive a food pellet reinforcer. Administration of the 5-HT_{1A} agonist 8-OH-DPAT reduces the control response rate (assessed by administration of vehicle placebo). The 5-HT_{1A} antagonist activity of a test compound is determined by measuring its ability to antagonize this agonist-induced decrease in response rate. A full antagonist effect is considered one in which the test compound completely reverses the agonist-induced response rate, returning it to control levels. Thus, this test can be used to evaluate test compounds for their ability to function as 5-HT_{1A} antagonists in vivo.

### OTHER EMBODIMENTS

The present invention may be embodied in other specific forms without departing from the spirit and essential attributes thereof and, accordingly, reference should be made to the appended claims, rather than to the foregoing specification, as indicating the scope of the invention.

What is claimed is:

1. A method for treating a cognitive disorder in a patient in need thereof, the method comprising administering to the patient synergistically effective amounts of a compound that is a 5-HT_{1A} antagonist and a cognitive enhancer.

2. The method of claim 1, wherein the cognitive disorder is dementia, Parkinson's disease, Huntington's disease, Alzheimer's disease, cognitive deficits associated with Alzheimer's disease, mild cognitive impairment, or schizophrenia.

3. The method of claim 1, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexanecarboxamide and pharmaceutically acceptable acid addition salts thereof,
(R)-N-(2-methyl-(4-indolyl-1-piperazinyl)ethyl)-N-(2-pyridinyl) cyclohexanecarboxamide and pharmaceutically acceptable acid addition salts thereof,
5-fluoro-8-(4-(4-(6-methoxyquinolin-8-yl)piperazin-1-yl)piperidin-1-yl)quinoline or a pharmaceutically acceptable acid addition salt thereof,
5-fluoro-4-methoxy-8-(4-(4-(6-methoxyquinolin-8-yl)piperazin-1-yl)piperidin-1-yl)-2-(trifluoromethyl)quinoline and pharmaceutically acceptable acid addition salts thereof,
6-methoxy-8-[4-(1-quinolin-8-yl-piperidin-4-yl)-piperazin-1-yl]-quinoline and pharmaceutically acceptable acid addition salts thereof,
6-fluoro-8-[4-(1-quinolin-8-yl-piperidin-4-yl)-piperazin-1-yl]-quinoline and pharmaceutically acceptable acid addition salts thereof,
8-{4-[4-(1H-indole-4-yl)-piperazin-1-yl]-piperidin-1-yl}-quinoline and pharmaceutically acceptable acid addition salts thereof,
5-fluoro-8-{4-[4-(5-fluoro-benzofuran-3-yl)-piperazin-1-yl]-piperidin-1-yl}-quinoline and pharmaceutically acceptable acid addition salts thereof,
7-fluoro-8-(4-(4-(6-methoxyquinolin-8-yl)piperazin-1-yl)piperidin-1-yl)quinoline and pharmaceutically acceptable acid addition salts thereof,
6-methoxy-8-(4-(1-(quinolin-8-ylmethyl)piperidin-4-yl)piperazin-1-yl)quinoline and pharmaceutically acceptable acid addition salts thereof,
8-[4-(1-quinolin-8-yl-piperidin-4-yl)-piperazin-1-yl]-5-trifluoromethyl-quinoline and pharmaceutically acceptable acid addition salts thereof,
5-methoxy-8-[4-(1-quinolin-8-yl-piperidin-4-yl)-piperazin-1-yl]-quinoline and pharmaceutically acceptable acid addition salts thereof,
5-fluoro-8-[4-(4-quinolin-8-yl-piperazin-1-yl)-piperidin-1-yl]-quinoline and pharmaceutically acceptable acid addition salts thereof, or
8-[4-(4-benzofuran-3-yl-piperazin-1-yl)-piperidin-1-yl]-6-chloro-quinoline and pharmaceutically acceptable acid addition salts thereof.

4. The method of claim 3, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexanecarboxamide and pharmaceutically acceptable acid addition salts thereof,
(R)-N-(2-methyl-(4-indolyl-1-piperazinyl)ethyl)-N-(2-pyridinyl) cyclohexanecarboxamide and pharmaceutically acceptable acid addition salts thereof,
5-fluoro-8-(4-(4-(6-methoxyquinolin-8-yl)piperazin-1-yl)piperidin-1-yl)quinoline or a pharmaceutically acceptable acid addition salt thereof,
5-fluoro-4-methoxy-8-(4-(4-(6-methoxyquinolin-8-yl)piperazin-1-yl)piperidin-1-yl)-2-(trifluoromethyl)quinoline and pharmaceutically acceptable acid addition salts thereof,

5. The method of claim 3, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexanecarboxamide and pharmaceutically acceptable acid addition salts thereof,
(R)-N-(2-methyl-(4-indolyl-1-piperazinyl)ethyl)-N-(2-pyridinyl) cyclohexanecarboxamide and pharmaceutically acceptable acid addition salts thereof,
6-methoxy-8-[4-(1-quinolin-8-yl-piperidin-4-yl)-piperazin-1-yl]-quinoline and pharmaceutically acceptable acid addition salts thereof, or
6-fluoro-8-[4-(1-quinolin-8-yl-piperidin-4-yl)-piperazin-1-yl]-quinoline and pharmaceutically acceptable acid addition salts thereof.

6. The method of claim 3, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexane carboxamide and pharmaceutically acceptable acid addition salts thereof,
(R)-N-(2-methyl-(4-indolyl-1-piperazinyl)ethyl)-N-(2-pyridinyl)cyclohexane carboxamide and pharmaceutically acceptable acid addition salts thereof, or
6-methoxy-8-[4-(1-quinolin-8-yl-piperidin-4-yl)-piperazin-1-yl]-quinoline and pharmaceutically acceptable acid addition salts thereof.

7. The method of claim 3, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof, or
(R)-N-(2-methyl-(4-indolyl-1-piperazinyl)ethyl)-N-(2-pyridinyl)cyclohexane carboxamide and pharmaceutically acceptable acid addition salts thereof.

8. The method of claim 3, wherein the 5-HT_{1A} antagonist compound is
(R)-N-(2-methyl-(4-indolyl-1-piperazinyl)ethyl)-N-(2-pyridinyl)cyclohexane carboxamide and pharmaceutically acceptable acid addition salts thereof.

9. The method of claim 1, wherein the cognitive enhancer is a cholinesterase inhibitor.

10. The method of claim 9, wherein the cholinesterase inhibitor is tacrine, donepezil, rivastigmine, or galantamine.

11. The method of claim 1, wherein the cognitive enhancer is an NMDA antagonist or an NMDA agonist.

12. The method of claim 1, wherein the cognitive enhancer is an ampakine class compound.

13. The method of claim 1, wherein the cognitive enhancer is a BZD/GABA receptor complex modulator.

14. The method of claim 1, wherein the cognitive enhancer is a serotonin antagonist.

15. The method of claim 1, wherein the cognitive enhancer is a nicotinic class compound.

16. The method of claim 1, wherein the cognitive enhancer is a muscarinic class compound.

17. The method of claim 1, wherein the cognitive enhancer is a MAO-B inhibitor.

18. The method of claim 1, wherein the cognitive enhancer is a PDE inhibitor.

19. The method of claim 1, wherein the cognitive enhancer is a G protein class compound.

20. The method of claim 1, wherein the cognitive enhancer is a channel modulator.

21. The method of claim 1, wherein the cognitive enhancer is an immunotherapeutic class compound.

22. The method of claim 1, wherein the cognitive enhancer is an anti-amyloid or amyloid lowering agent.

23. The method of claim 1, wherein the cognitive enhancer is a statin or a PPARS modulator.

24. The method of claim 1, wherein the method comprises oral delivery of the compound that is a 5-HT_{1A} antagonist.

25. The method of claim 1, wherein the method comprises delivery of a sustained release compound.

26. A method of enhancing cognition in a patient in need thereof, the method comprising administering to the patient synergistically effective amounts of a 5-HT_{1A} antagonist compound and a cognitive enhancer.

27. A pharmaceutical composition for treating a cognitive disorder, the composition comprising a 5-HT_{1A} antagonist compound and a cognitive enhancer.

28. The pharmaceutical composition of claim 27, wherein the 5-HT_{1A} antagonist compound is
(R)-4-cyano-N-{2-[4-(2,3-dihydro-benzo[1,4]dioxin-5-yl)-piperazin 1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexanecarboxamide and pharmaceutically acceptable acid addition salts thereof,
(R)-N-(2-methyl-(4-indolyl-1-piperazinyl)ethyl)-N-(2-pyridinyl) cyclohexanecarboxamide and pharmaceutically acceptable acid addition salts thereof,
5-fluoro-8-(4-(4-(6-methoxyquinolin-8-yl)piperazin-1-yl)piperidin-1-yl)quinoline or a pharmaceutically acceptable acid addition salt thereof,
5-fluoro-4-methoxy-8-(4-(4-(6-methoxyquinolin-8-yl)piperazin-1-yl)piperidin-1-yl)-2-(trifluoromethyl)quinoline and pharmaceutically acceptable acid addition salts thereof,
6-methoxy-8-[4-(1-quinolin-8-yl-piperidin-4-yl)-piperazin-1-yl]-quinoline and pharmaceutically acceptable acid addition salts thereof,
6-fluoro-8-[4-(1-quinolin-8-yl-piperidin-4-yl)-piperazin-1-yl]-quinoline and pharmaceutically acceptable acid addition salts thereof,
8-{4-[4-(1H-indole-4-yl)-piperazin-1-yl]-piperidin-1-yl}-quinoline and pharmaceutically acceptable acid addition salts thereof,
5-fluoro-8-{4-[4-(5-fluoro-benzofuran-3-yl)-piperazin-1-yl]- piperidin-1-yl}-quinoline and pharmaceutically acceptable acid addition salts thereof,
7-fluoro-8-(4-(4-(6-methoxyquinolin-8-yl)piperazin-1-yl)piperidin-1-yl)quinoline and pharmaceutically acceptable acid addition salts thereof,
6-methoxy-8-(4-(1-(quinolin-8-ylmethyl)piperidin-4-yl)piperazin-1-yl)quinoline and pharmaceutically acceptable acid addition salts thereof,
8-[4-(1-quinolin-8-yl-piperidin-4-yl)-piperazin-1-yl]-5-trifluoromethyl-quinoline and pharmaceutically acceptable acid addition salts thereof,
5-methoxy-8-[4-(1-quinolin-8-yl-piperidin-4-yl)-piperazin-1-yl]-quinoline and pharmaceutically acceptable acid addition salts thereof,
5-fluoro-8-[4-(4-quinolin-8-yl-piperazin-1-yl)-piperidin-1-yl]-quinoline and pharmaceutically acceptable acid addition salts thereof, or
8-[4-(4-benzofuran-3-yl-piperazin-1-yl)-piperidin-1-yl]-6-chloro-quinoline and pharmaceutically acceptable acid addition salts thereof.

29. The pharmaceutical composition of claim 27, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexanecarboxamide and pharmaceutically acceptable acid addition salts thereof,
(R)-N-(2-methyl-(4-indolyl-1-piperazinyl)ethyl)-N-(2-pyridinyl) cyclohexanecarboxamide and pharmaceutically acceptable acid addition salts thereof,
6-methoxy-8-[4-(1-quinolin-8-yl-piperidin-4-yl)-piperazin-1-yl]-quinoline and pharmaceutically acceptable acid addition salts thereof, or
6-fluoro-8-[4-(1-quinolin-8-yl-piperidin-4-yl)-piperazin-1-yl]-quinoline and pharmaceutically acceptable acid addition salts thereof.

30. The pharmaceutical composition of claim 27, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof,
N-[2-[4-(2-methoxyphenyl)-1-piperazinyl]ethyl]-N-(2-pyridinyl)cyclohexane carboxamide and pharmaceutically acceptable acid addition salts thereof,
(R)-N-(2-methyl-(4-indolyl-1-piperazinyl)ethyl)-N-(2-pyridinyl)cyclohexane carboxamide and pharmaceutically acceptable acid addition salts thereof, or
6-methoxy-8-[4-(1-quinolin-8-yl-piperidin-4-yl)-piperazin-1-yl]-quinoline and pharmaceutically acceptable acid addition salts thereof.

31. The pharmaceutical composition of claim 27, wherein the 5-HT_{1A} antagonist compound is
(R)-4-cyano-N-{2-[4-(2,3-dihydro-benzo[1,4]dioxin-5-yl)-piperazin-1-yl]propyl}-N-pyridin-2-yl-benzamide and pharmaceutically acceptable acid addition salts thereof, or
(R)-N-(2-methyl-(4-indolyl-1-piperazinyl)ethyl)-N-(2-pyridinyl)cyclohexane carboxamide and pharmaceutically acceptable acid addition salts thereof.

32. The pharmaceutical composition of claim 27, wherein the 5-HT_{1A} antagonist compound is
(R)-N-(2-methyl-(4-indolyl-1-piperazinyl)ethyl)-N-(2-pyridinyl) cyclohexane carboxamide and pharmaceutically acceptable acid addition salts thereof.

33. The pharmaceutical composition of claim 27, wherein the cognitive enhancer is a cholinesterase inhibitor.

34. The pharmaceutical composition of claim 33, wherein the cholinesterase inhibitor is tacrine, donepezil, rivastigmine, or galantamine.

35. The pharmaceutical composition of claim 27, wherein the cognitive enhancer is an NMDA antagonist or an NMDA agonist.

36. The pharmaceutical composition of claim 27, wherein the cognitive enhancer is an ampakine class compound.

37. The pharmaceutical composition of claim 27, wherein the cognitive enhancer is a BZD/GABA receptor complex modulator.

38. The pharmaceutical composition of claim 27, wherein the cognitive enhancer is a serotonin antagonist.

39. The pharmaceutical composition of claim 27, wherein the cognitive enhancer is a nicotinic class compound.

40. The pharmaceutical composition of claim 27, wherein the cognitive enhancer is a muscarinic class compound.

41. The pharmaceutical composition of claim 27, wherein the cognitive enhancer is a MAO-B inhibitor.

42. The pharmaceutical composition of claim 27, wherein the cognitive enhancer is a PDE inhibitor.

43. The pharmaceutical composition of claim 27, wherein the cognitive enhancer is a G protein class compound.

44. The pharmaceutical composition of claim 27, wherein the cognitive enhancer is a channel modulator.

45. The pharmaceutical composition of claim 27, wherein the cognitive enhancer is an immunotherapeutic class compound.

46. The pharmaceutical composition of claim 27, wherein the cognitive enhancer is an anti-amyloid or amyloid lowering agent.

47. The pharmaceutical composition of claim 27, wherein the cognitive enhancer is a statin or a PPARS modulator.

48. The pharmaceutical composition of claim 27, wherein the pharmaceutical composition comprises a formulation suitable for oral delivery.

49. The pharmaceutical composition of claim 27, wherein the pharmaceutical composition comprises a formulation suitable for sustained release.

50. The pharmaceutical composition of claim 27, wherein the 5-HT_{1A} antagonist compound and the cognitive enhancer are present in synergistically effective amounts.

51. A package comprising a 5-HT_{1A} antagonist and a cognitive enhancer, wherein the instructions comprise instructions for treating a cognitive disorder.

52. A pharmaceutical product containing a 5-HT_{1A} antagonist and a cognitive enhancer as a combined preparation for simultaneous, separate or sequential use in therapy for treating a cognitive disorder.
